(19) United States
(12) Patent Application Publication
(10) Pub. No.: US 2010/0227974 A1
(43) Pub. Date: Sep. 9, 2010

(54) CONJUGATED POLYMERS, PROCESS FOR THEIR PREPARATION AND THEIR USE

(76) Inventors: Niels Schulte, Kelkheim (DE); René Scheurich, Gross-Zimmern (DE); Junyou Pan, Frankfurt (DE)

Correspondence Address: CONNOLLY BOVE LODGE & HUTZ, LLP, P O BOX 2207, WILMINGTON, DE, 19899, US

(21) Appl. No.: 12/377,628

(22) PCT Filed: Jul. 18, 2007
(86) PCT No.: PCT/EP07/06383

§ 371 Date: Feb. 16, 2009

(30) Foreign Application Priority Data
Aug. 17, 2006 (DE) 10 2006 038 683.3

(51) Int. Cl. C08L 85/04; C08G 79/08; C07C 25/22

(52) U.S. Cl. 524/610; 528/8; 525/389; 570/183

(57) ABSTRACT

The present invention relates to conjugated polymers and dendrimers containing dihydrophenanthrene structural units, to processes for the preparation thereof, to the use thereof in electronic components, in particular in polymeric organic light-emitting diodes, to monomers for the preparation thereof, and to components and light-emitting diodes comprising polymers and dendrimers of this type.

The present invention relates to conjugated polymers and dendrimers containing dihydrophenanthrene structural units, to processes for the preparation thereof, to the use thereof in electronic components, in particular in polymeric organic light-emitting diodes, to monomers for the preparation thereof, and to components and light-emitting diodes comprising polymers and dendrimers of this type.

Conjugated polymers are currently being intensively investigated as highly promising materials in PLEDs (polymeric light emitting diodes). Their simple processing in contrast to SMOLEDs (small molecule organic light emitting diodes) promises less expensive production of corresponding light-emitting diodes.

Since PLEDs usually consist only of a light-emitting layer, polymers are required which are able to combine all the functions of an OLED (charge injection, charge transport, recombination). A very wide variety of monomers which undertake corresponding functions are therefore employed during the polymerisation.

In contrast to PLEDs, SMOLEDs are constructed from a plurality of layers which fulfil the various functions. Here too, a light-emitting layer which comprises the emitter is present.

In PLEDs, certain comonomers are usually copolymerised into the corresponding polymers in order to produce all three emission colours (cf., for example, WO 00/46321, WO 03/020790 and WO 02/077060). It is then generally possible—starting from a blue-emitting base polymer (“backbone”)—to produce the two other primary colours red and green.

The most important criteria of an OLED are efficiency, colour, lifetime and processability. These properties are crucially determined by the combination of backbone and emitter.

The lifetime depends on the backbone stability, which is in turn influenced by the charge-carrier transport, in particular the electron transport.

In accordance with the prior art, conjugated polymers based on fluorenes, indenofluorenes, spirobifluorenes, phenanthrenes and dihydrophenanthrenes, in particular, are synthesised today in order to be able to produce blue-luminescent organic light-emitting diodes. Two-layer structures, in which an emission layer is applied to an injection layer, are increasingly finding acceptance here.

Polymers containing dihydrophenanthrenes are described, for example, in WO 05/14689 A2 and EP 1 074 600 A2.

However, the systems described above have deficiencies in relation to the following parameters or properties:
The lifetime of the blue-emitting polymers is by far not yet sufficient for use in mass products.
The efficiency of the polymers prepared in accordance with the prior art is too low.
The operating voltages are too high for the potential applications.
The materials frequently suffer from a shift in the emission characteristics during operation.
The materials can often only be processed with difficulty in the OLED production process and result, for example in the case of processing in solution (for example ink-jet printing), in an increase in the viscosity.

Surprisingly, it has now been found that polymers containing dihydrophenanthrene units in accordance with the present invention exhibit significantly improved colour stability and significantly improved electron stability and consequently a smaller increase in operating voltage. This enables the lifetime of the polymers in PLEDs to be significantly increased.

The invention thus relates to conjugated polymers and dendrimers which are characterised in that they contain one or more units of the formula (1)

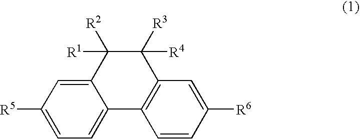

in which
R¹⁻⁴ on each occurrence, identically or differently, denote H, F or a straight-chain, branched or cyclic alkyl, alkenyl or alkynyl group, in which, in addition, one or more non-adjacent C atoms may be replaced by O, S, CO—O or O—CO—O and in which, in addition, one or more H atoms may be replaced by fluorine, or an aryl, aralkyl, aralkenyl, aralkynyl or heteroaryl group, which may also be mono- or polysubstituted, where two or more radicals R¹⁻⁴ may also form with one another an aliphatic or aromatic, mono- or polycyclic ring system, which may also form, with the dihydrophenanthrene structure, a condensed or spiro-linked ring system,
   where at least two of the radicals R¹⁻⁴ are different from H,
R^{5,6} denote a link in the polymer or dendrimer or a reactive group which is suitable for a polymerisation reaction.

The linking of the units of the formula (1) to adjacent units in the polymers according to the invention can take place along the polymer main chain or also in the polymer side chain.

Preferably three, particularly preferably all four, radicals R¹⁻⁴ are different from H.

Particularly preferred radicals R¹⁻⁴ are straight-chain, branched or cyclic alkyl, alkenyl or alkynyl having 1 to 40, preferably 1 to 25, particularly preferably 1 to 18, C atoms, optionally substituted aryl having 5 to 40, preferably 5 to 25, C atoms, or optionally substituted alkylaryl, arylalkyl having 5 to 40, preferably 5 to 25, C atoms.

Preference is furthermore given to units of the formula (1) in which two or more radicals R¹⁻⁴, particularly preferably both radicals R¹ and R² and/or both radicals R³ and R⁴, form an aliphatic or aromatic, mono- or polycyclic ring system. Preferred ring systems of this type are the aryl and heteroaryl groups mentioned below. The ring systems may also be condensed with or spiro-linked to the dihydrophenanthrene structure from formula (1). Preferred compounds of this type are, for example, those in which the two radicals R¹ and R² or the two radicals R³ and R⁴ form an optionally substituted fluorene group, which is spiro-linked via its 9-position to the 9- or 10-position of the dihydrophenanthrene structure in formula (1).

Very particularly preferred carbon and hydrocarbon radicals are C₁-C₄₀ alkyl, C₂-C₄₀ alkenyl, C₂-C₄₀ alkynyl, C₃-C₄₀ alkyl, C₄-C₄₀ alkyldienyl, C₄-C₄₀ polyenyl, C₆-C₄₀ aryl, C₆-C₄₀ alkylaryl, C₆-C₄₀ arylalkyl, C₆-C₄₀ heteroaryl, C₃-C₄₀ cycloalkyl and C₃-C₄₀ cycloalkenyl. Particular preference is given to C₁-C₂₂ alkyl, C₂-C₂₂ alkenyl, C₂-C₂₂ alkynyl, C₃-C₂₂ alkyl, C₄-C₂₂ alkyldienyl, C₆-C₁₂ aryl, C₆-C₂₀ arylalkyl and C₆-C₂₀ heteroaryl.

Preferred alkyl groups are, for example, methyl, ethyl, n-propyl, isopropyl, cyclopropyl, n-butyl, isobutyl, s-butyl, t-butyl, 2-methylbutyl, cyclobutyl, n-pentyl, s-pentyl, cyclopentyl, n-hexyl, cyclohexyl, 2-ethylhexyl, n-heptyl, cycloheptyl, 1,1,5-trimethylheptyl, n-octyl, cyclooctyl, dodecanyl, trifluoromethyl, perfluoro-n-butyl, 2,2,2-trifluoroethyl, perfluorooctyl and perfluorohexyl.

Preferred alkenyl groups are, for example, ethenyl, propenyl, butenyl, pentenyl, cyclopentenyl, hexenyl, cyclohexenyl, heptenyl, cycloheptenyl, octenyl and cyclooctenyl.

Preferred alkynyl groups are, for example, ethynyl, propynyl, butynyl, pentynyl, hexynyl and octynyl.

Aryl groups may be monocyclic or polycyclic, i.e. they may have one ring (for example phenyl) or two or more rings, which may also be condensed (for example naphthyl) or covalently linked (for example biphenyl), or contain a combination of condensed and linked rings. Preference is given to fully conjugated aryl groups.

Preferred aryl groups are, for example, phenyl, biphenyl, triphenyl, 1,1′:3′,1″-terphenyl-2′-yl, naphthyl, anthracene, binaphthyl, phenanthrene, pyrene, dihydropyrene, chrysene, perylene, tetracene, pentacene, benzopyrene, fluorene, indene, indenofluorene and spirobifluorene.

Preferred heteroaryl groups are, for example, 5-membered rings, such as pyrrole, pyrazole, imidazole, 1,2,3-triazole, 1,2,4-triazole, tetrazole, furan, thiophene, selenophene, oxazole, isoxazole, 1,2-thiazole, 1,3-thiazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole and 1,3,4-thiadiazole, 6-membered rings, such as pyridine, pyridazine, pyrimidine, pyrazine, 1,3,5-triazine, 1,2,4-triazine, 1,2,3-triazine, 1,2,4,5-tetrazine, 1,2,3,4-tetrazine and 1,2,3,5-tetrazine, or condensed groups, such as indole, isoindole, indolizine, indazole, benzimidazole, benzotriazole, purine, naphthimidazole, phenanthrimidazole, pyridimidazole, pyrazinimidazole, quinoxalinimidazole, benzoxazole, naphthoxazole, anthroxazole, phenanthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquinoline, acridine, phenothiazine, phenoxazine, benzopyridazine, benzopyrimidine, quinoxaline, phenazine, naphthyridine, azacarbazole, benzocarboline, phenanthridine, phenanthroline, thieno[2,3b]thiophene, thieno[3,2b]thiophene, dithienothiophene, isobenzothiophene, dibenzothiophene, benzothiadiazothiophene or combinations of these groups. The heteroaryl groups may also be substituted by alkyl, alkoxy, thioalkyl, fluorine, fluoroalkyl or other aryl or heteroaryl groups.

The groups R¹⁻⁴ optionally have one or more substituents L, which are preferably selected from the group comprising silyl, sulfo, sulfonyl, formyl, keto, amine, imine, nitrile, mercapto, nitro, halogen, phosphine oxide, C₁₋₁₂ alkyl, C₁₋₁₂ fluoroalkyl, C₆₋₁₂ aryl or combinations of these groups.

Preferred substituents L are, for example, solubility-promoting groups, such as alkyl, electron-withdrawing groups, such as fluorine, nitro or nitrile, or substituents for increasing the glass transition temperature (Tg) in the polymer, in particular bulky groups, such as, for example, t-butyl or optionally substituted aryl groups.

Further preferred substituents L are, for example, F, Cl, Br, I, —CN, —NO₂, —NCO, —NCS, —OCN, —SCN, —C(═O)NR₂, —C(═O)X, —C(═O)R, —NR₂, —P(O)R₂, optionally substituted silyl, aryl having 4 to 40, preferably 6 to 20, C atoms, and straight-chain or branched alkyl or fluoroalkyl having 1 to 22 C atoms, in which one or more H atoms may optionally be replaced by F or Cl. X denotes halogen. R on each occurrence, identically or differently, denotes H, a straight-chain, branched or cyclic alkyl, alkenyl, alkynyl or alkoxy chain having 1 to 22 C atoms, in which, in addition, one or more non-adjacent C atoms may be replaced by O, S, CO—O or O—CO—O, where, in addition, one or more H atoms may be replaced by fluorine, or an optionally substituted aryl or aryloxy group having 5 to 40 C atoms, in which, in addition, one or more C atoms may be replaced by O, S or N.

The terms “alkyl”, “aryl”, “heteroaryl”, etc., also encompass polyvalent groups, for example alkylene, arylene, heteroarylene, etc.

“Halogen” denotes F, Cl, Br or I.

For the purposes of this invention, “conjugated polymers” are polymers which contain principally sp²-hybridised (or optionally also sp-hybridised) carbon atoms, which may also be replaced by corresponding heteroatoms, in the main chain. In the simplest case, this means the alternating presence of double and single bonds in the main chain, but also polymers containing units such as, for example, meta-linked phenylene are intended to be regarded as conjugated polymers for the purposes of this invention. “Principally” means that naturally (randomly) occurring defects which result in conjugation interruptions do not devalue the term “conjugated polymer”. Furthermore, the term “conjugated” is likewise used in this application text if the main chain contains, for example, arylamine units, arylphosphine units, arylphosphine oxide units and/or certain heterocycles (i.e. conjugation via N, O, P or S atoms) and/or organometallic complexes (i.e. conjugation via the metal atom). An analogous situation applies to conjugated dendrimers.

The term “dendrimer” here is intended to be taken to mean a highly branched compound which is built up from a multifunctional centre (core) to which branched monomers are bonded in a regular construction, giving a tree-like structure. Both the core and also the monomers here can adopt any desired branched structures which consist both of purely organic units and also organometallic compounds or coordination compounds. “Dendrimer” here is in general intended to be understood as described, for example, by M. Fischer and F. Vögtle (Angew. Chem., Int. Ed. 1999, 38, 885).

The units of the formula (1) can be incorporated in accordance with the invention into the main or side chain of the polymer. In the case of incorporation into the side chain, it is possible for the unit of the formula (1) to be in conjugation with the polymer main chain or to be non-conjugated with the polymer main chain.

In a preferred embodiment of the invention, the unit of the formula (1) is in conjugation with the polymer main chain. This can be achieved on the one hand by incorporating this unit into the main chain of the polymer in such a way that the conjugation of the polymer, as described above, is thereby retained. On the other hand, this unit can also be linked into the side chain of the polymer in such a way that conjugation with the main chain of the polymer exists. This is the case, for example, if the linking to the main chain takes place only via sp²-hybridised (or optionally also via sp-hybridised) carbon atoms, which may also be replaced by corresponding heteroatoms. However, if the linking takes place through units such as, for example, simple (thio)ether bridges, esters, amides or alkylene chains, the structural unit of the formula (1) is defined as non-conjugated with the main chain.

The linking of the units of the formula (1) to the main chain can take place directly or via one or more additional units. Preferred units for the linking are optionally substituted, straight-chain, branched or cyclic alkylene groups, alkenylene groups or alkynylene groups, in particular optionally substituted C═C double bonds, C≡C triple bonds, or aromatic units, further di- and triarylamino units, arylenevinylene units or aryleneethynylene units which are identical to or different from formula (1). Preference is given to linking in conjugation with the main chain.

The radicals R¹⁻⁴ in formula (1) are preferably selected from the above-mentioned groups.

Particular preference is given to structural units selected from the following sub-formulae:

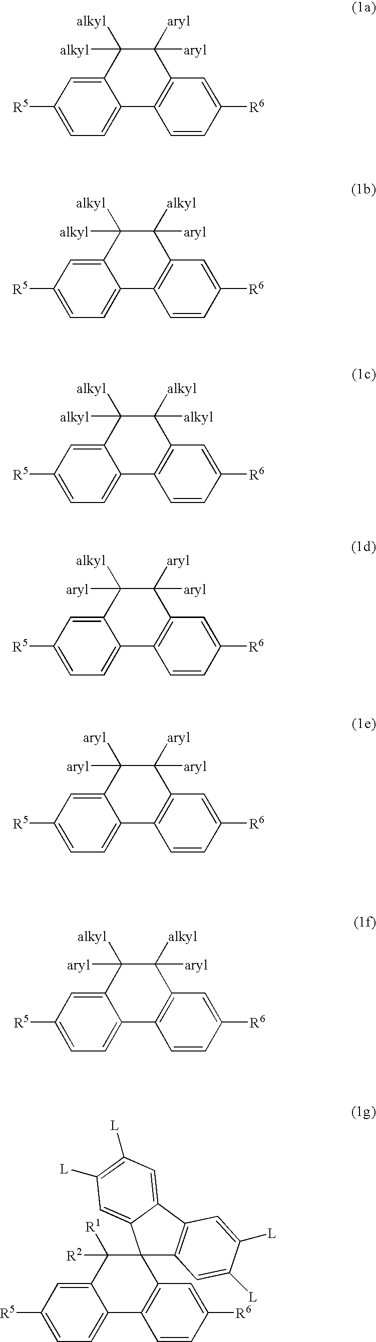

in which R¹, R², R⁵ and R⁶ have the meaning indicated in formula (1), “alkyl” on each occurrence, identically or differently, denotes a straight-chain, branched or cyclic alkyl radical having 1 to 20 C atoms, “aryl” on each occurrence, identically or differently, denotes an optionally substituted aryl radical having 5 to 20 C atoms or heteroaryl radical having 3 to 20 C atoms, and L on each occurrence, identically or differently, denotes a substituent as indicated above.

The structural units of the formula (1) are readily accessible in high yields.

The conjugated polymers and dendrimers according to the invention preferably contain at least 1 mol %, particularly preferably 10 to 100 mol %, and in particular 10 to 99 mol %, of one or more units of the formula (1).

Particular preference is given to polymers according to the invention which also contain further structural elements in addition to units of the formula (1) and should thus be regarded as copolymers. Although the further structural units are necessary for the synthesis of the copolymers according to the invention, they are, however, not themselves a subject-matter of the present invention and should thus be described by reference. Reference should also be made here, in particular, to the relatively extensive lists in WO 02/077060, WO 2005/014689 and the references cited in these specifications. These further structural units can originate, for example, from the classes described below:
Group 1: structural units which represent the polymer backbone.
Group 2: structural units which enhance the hole-injection and/or -transport properties of the polymers.
Group 3: structural units which significantly enhance the electron-injection and/or -transport properties of the polymers.
Group 4: structural units which have combinations of individual units from group 2 and group 3.
Group 5: structural units which influence the morphology and/or emission colour of the resultant polymers.
Group 6: structural units which modify the emission characteristics to such an extent that electrophosphorescence can be obtained instead of electrofluorescence.
Group 7: structural units which improve the transfer from the singlet state to the triplet state.

Suitable and preferred units for the above-mentioned groups are described below.

Group 1—Structural Units which Represent the Polymer Backbone:

Preferred units from group 1, besides the units of the formula (1), are, in particular, those which contain aromatic or carbocyclic structures having 6 to 40 C atoms. Suitable and preferred units are, inter alia, fluorene derivatives, as disclosed, for example, in EP 0842208, WO 99/54385, WO 00/22027, WO 00/22026 and WO 00/46321, indenofluorenes, furthermore spirobifluorene derivatives, as disclosed, for example, in EP 0707020, EP 0894107 and WO 03/020790, or dihydrophenanthrene derivatives, as disclosed, for example, in WO 2005/014689. It is also possible to use a combination of two or more of these monomer units, as described, for example, in WO 02/077060. Preferred units for the polymer backbone, besides the units of the formula (1), are, in particular, spirobifluorenes and indenofluorenes.

Particularly preferred units from group 1 are divalent units of the following formulae, in which the dashed line denotes the link to the adjacent unit:

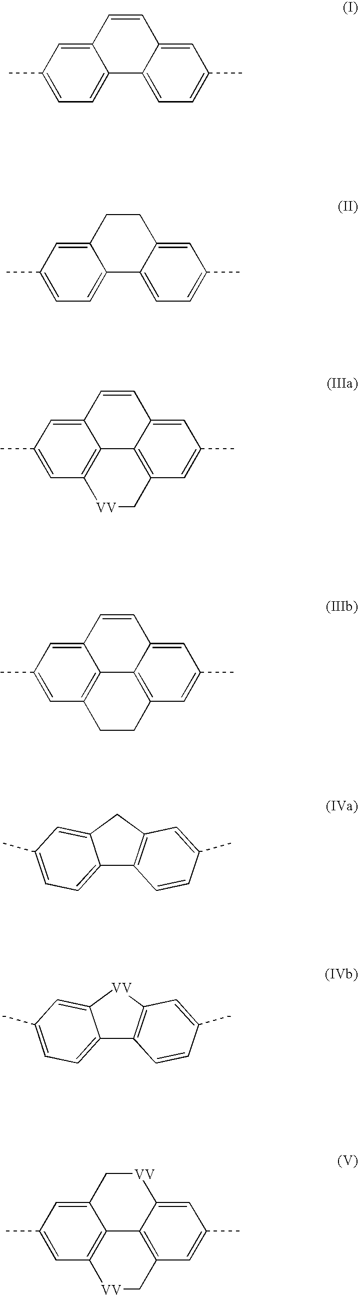

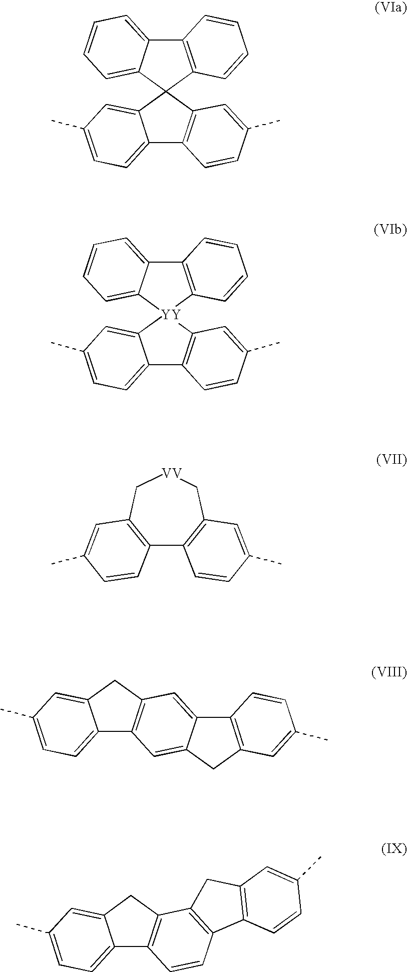

in which the individual radicals have the following meaning:

### YY is Si or Ge,

### VV is O, S or Se,

and where the various formulae may also additionally be substituted in the free positions by one or more substituents R¹¹, and R¹¹ denotes the following:
R¹¹ is on each occurrence, identically or differently, H, a straight-chain, branched or cyclic alkyl or alkoxy chain having 1 to 22 C atoms, in which, in addition, one or more non-adjacent C atoms may be replaced by O, S, CO—O or O—CO—O, where, in addition, one or more H atoms may be replaced by fluorine, an aryl or aryloxy group having 5 to 40 C atoms, in which, in addition, one or more C atoms may be replaced by O, S or N and which may also be substituted by one or more non-aromatic radicals R¹², or F, CN, N(R¹²)₂ or B(R¹²)₂; and
R¹² is on each occurrence, identically or differently, H, a straight-chain, branched or cyclic alkyl chain having 1 to 22 C atoms, in which, in addition, one or more non-adjacent C atoms may be replaced by O, S, CO—O or O—CO—O, where, in addition, one or more H atoms may be replaced by fluorine, or an optionally substituted aryl group having 5 to 40 C atoms, in which, in addition, one or more C atoms may be replaced by O, S or N.
   Group 2—Structural Units which Enhance the Hole-Injection and/or -Transport Properties of the Polymers:

These are generally aromatic amines or electron-rich heterocycles, such as, for example, substituted or unsubstituted triarylamines, benzidines, tetraarylene-para-phenylenediamines, phenothiazines, phenoxazines, dihydrophenazines, thianthrenes, dibenzo-p-dioxins, phenoxathiynes, carbazoles, azulenes, thiophenes, pyrroles, furans and further O-, S- or N-containing heterocycles having a high HOMO (HOMO=highest occupied molecular orbital). However, triaryiphosphines, as described, for example, in WO 2005/017065 A1, are also suitable here.

Particularly preferred units from group 2 are divalent units of the following formulae, in which the dashed line denotes the link to the adjacent unit:

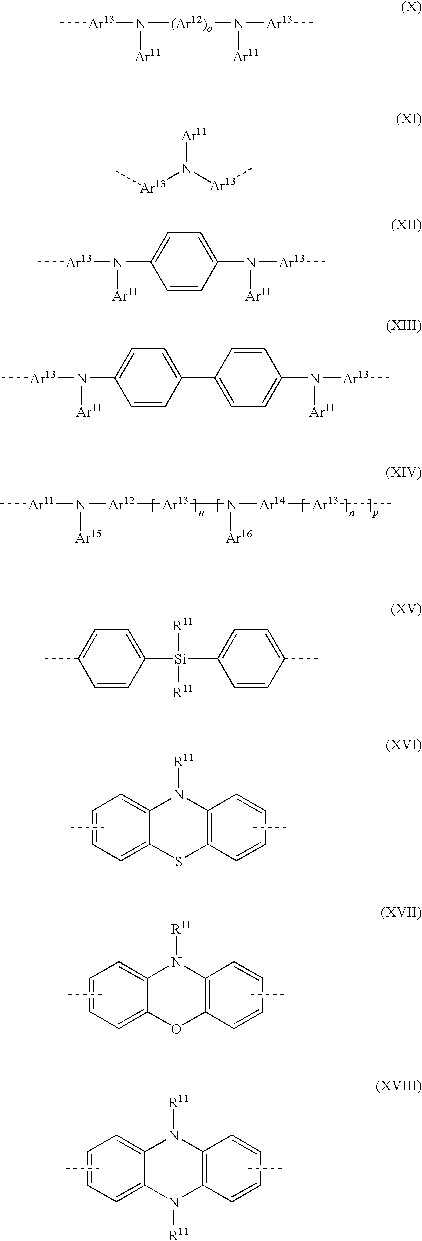

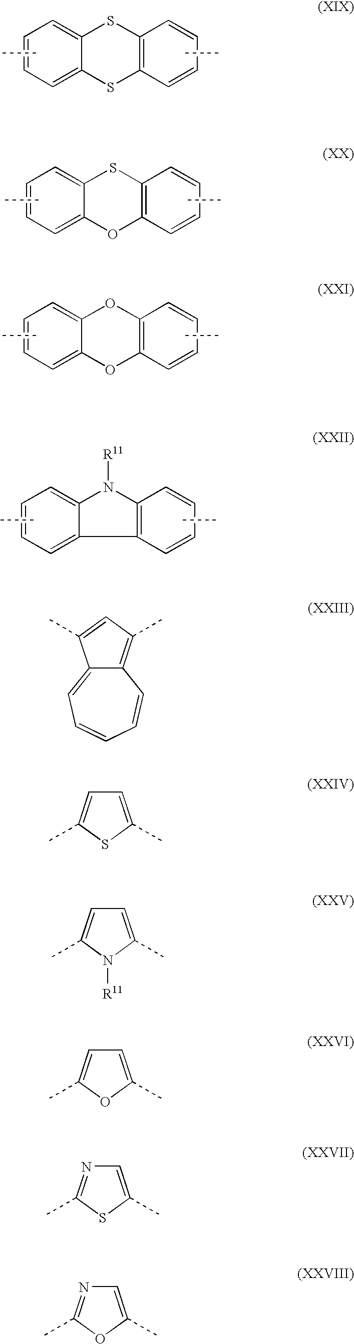

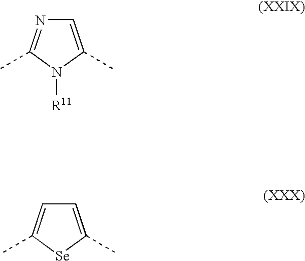

where R¹¹ has one of the meanings indicated above, the various formulae may also additionally be substituted in the free positions by one or more substituents R¹¹, and the symbols and indices have the following meanings:
n is, identically or differently on each occurrence, 0, 1 or 2,
p is, identically or differently on each occurrence, 0, 1 or 2, preferably 0 or 1,
o is, identically or differently on each occurrence, 1, 2 or 3, preferably 1 or 2,
Ar¹¹, Ar¹³ are on each occurrence, identically or differently, an aromatic or heteroaromatic ring system having 2 to 40 C atoms, which may be mono- or polysubstituted by R¹¹ or also unsubstituted; the possible substituents R¹¹ here can potentially be in any free position,
Ar¹², Ar¹⁴ are on each occurrence, identically or differently, Ar¹¹, Ar¹³ or a substituted or unsubstituted stilbenzylene or tolanylene unit,
Ar¹⁵ is, identically or differently on each occurrence, either a system as described by Ar¹¹ or an aromatic or heteroaromatic ring system having 9 to 40 aromatic atoms (C or heteroatoms), which may be mono- or polysubstituted by R¹¹ or unsubstituted and which consists of at least two condensed rings; the possible substituents R¹¹ here can potentially be in any free position.
   Group 3—Structural Units which Significantly Enhance the Electron-Injection and/or -Transport Properties of the Polymers:

These are generally electron-deficient aromatics or heterocycles, such as, for example, substituted or unsubstituted pyridines, pyrimidines, pyridazines, pyrazines, anthracenes, oxadiazoles, quinolines, quinoxalines, phenazines, ketones, phosphine oxides, sulfoxides or triazines, but also compounds such as triarylboranes and further O-, S- or N-containing heterocycles having a low LUMO (LUMO=lowest unoccupied molecular orbital), and benzophenones and derivatives thereof, as disclosed, for example, in WO 05/040302.

Particularly preferred units from group 3 are divalent units of the following formulae, in which the dashed line denotes the link to the adjacent unit:

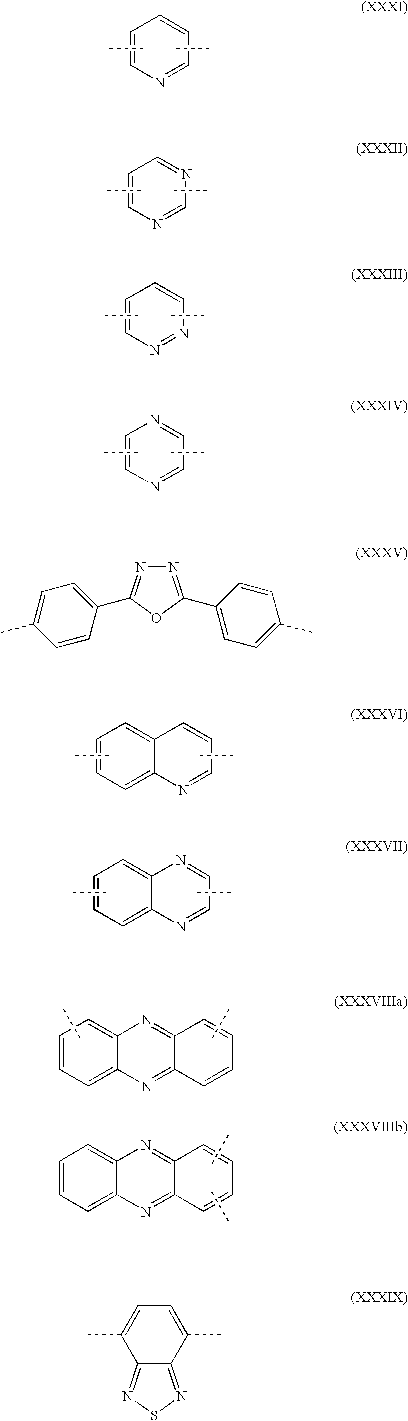

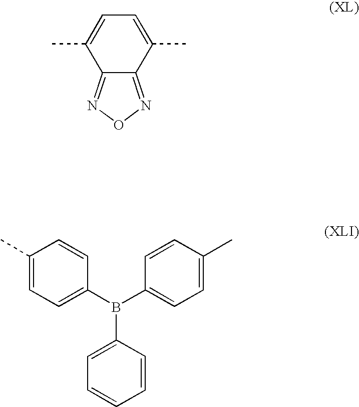

where the various formulae may be substituted in the free positions by one or more substituents R¹¹ as defined above.
Group 4—Structural Units which have Combinations of Individual Units from Group 2 and Group 3:

It is also possible for the polymers according to the invention to contain units in which structures which increase the hole mobility and the electron mobility are bonded directly to one another. However, some of these units shift the emission colour into the yellow or red. Their use in the polymers according to the invention for generating blue or green emission is therefore less preferred.

If such units from group 4 are present in the polymers according to the invention, they are preferably selected from divalent units of the following formulae, in which the dashed line denotes the link to the adjacent unit:

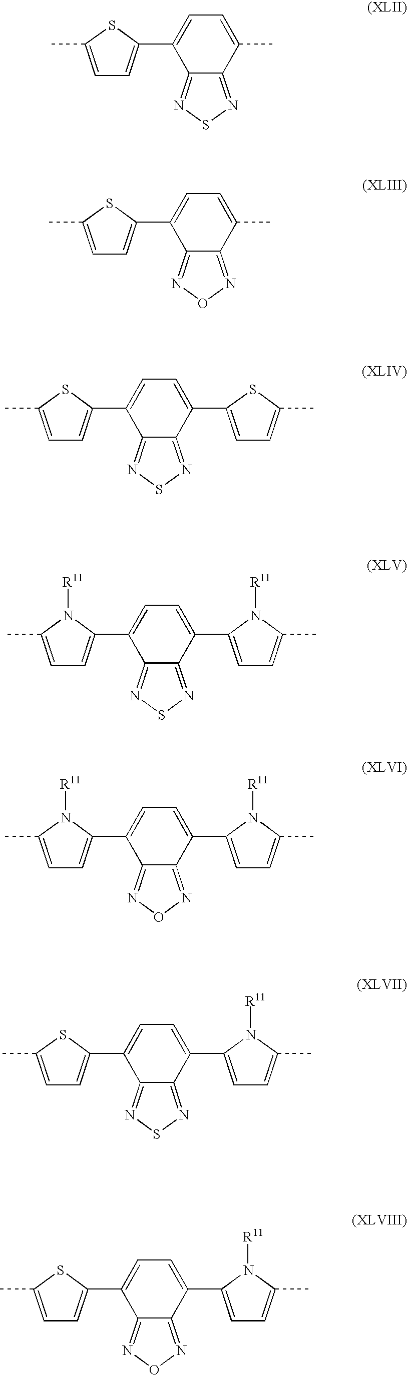

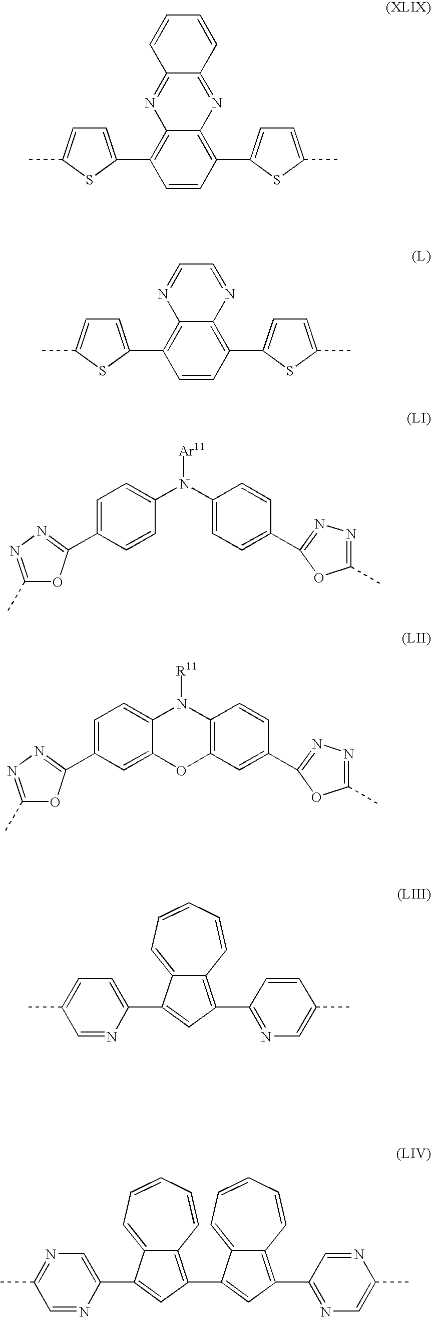

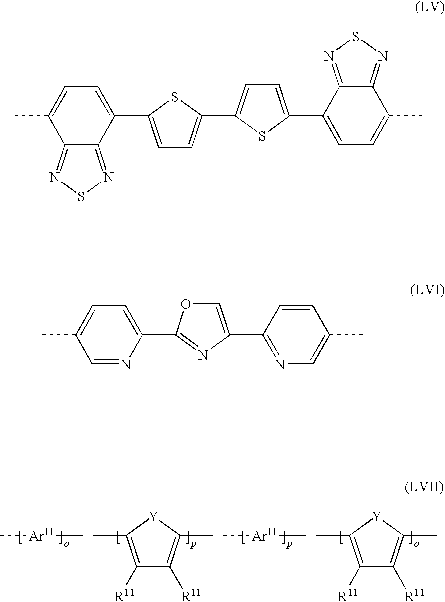

where the various formulae may be substituted in the free positions by one or more substituents R¹¹, the symbols R¹¹, Ar¹¹, p and o have the above-mentioned meaning, and Y is on each occurrence, identically or differently, O, S, Se, N, P, Si or Ge.
Group 5—Structural Units which Influence the Morphology and/or Emission Colour of the Resultant Polymers:

Besides the units mentioned above, these are those which have at least one further aromatic or another conjugated structure which does not fall under the above-mentioned groups, i.e. which has only little effect on the charge-carrier mobility, which are not organometallic complexes or which have no influence on the singlet-triplet transfer. Structural elements of this type may influence the morphology, but also the emission colour of the resultant polymers. Depending on the unit, they can therefore also be employed as emitters. Preference is given here to substituted or unsubstituted aromatic structures having 6 to 40 C atoms or also tolan, stilbene or bisstyrylarylene derivatives, each of which may be substituted by one or more radicals R¹¹. Particular preference is given here to the incorporation of 1,4-phenylene, 1,4-naphthylene, 1,4- or 9,10-anthrylene, 1,6- or 2,7- or 4,9-pyrenylene, 3,9- or 3,10-perylenylene, 4,4′-biphenylylene, 4,4″-terphenylylene, 4,4′-bi-1,1′-naphthylylene, 4,4′-tolanylene, 4,4′-stilbenzylene or 4,4″-bisstyrylarylene derivatives.

Very particular preference is given to substituted or unsubstituted structures of the following formulae:

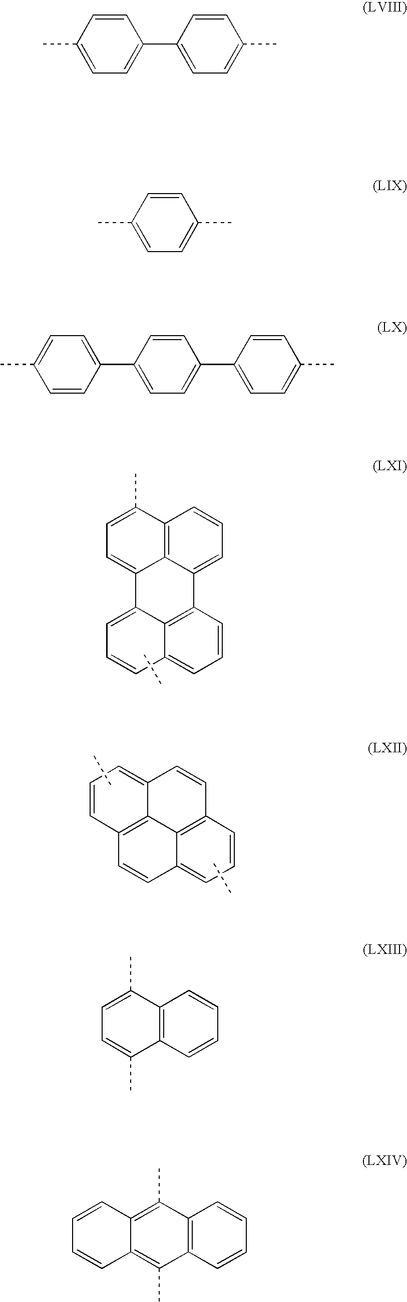

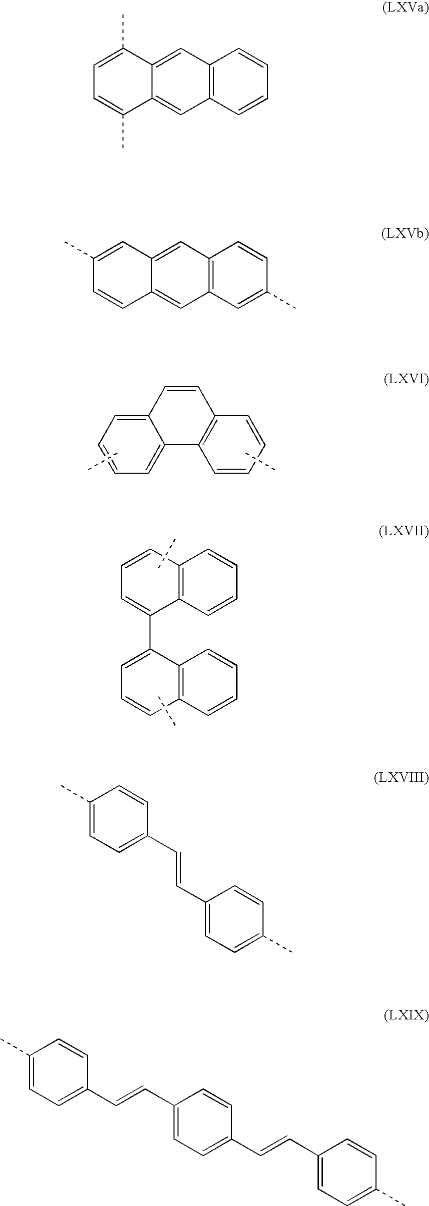

where the various formulae may be substituted in the free positions by one or more substituents R¹¹ as defined above.
Group 6—Structural Units which Modify the Emission Characteristics to such an Extent that Electrophosphorescence can be Obtained Instead of Electrofluorescence:

These are, in particular, those units which are able to emit light from the triplet state with high efficiency even at room temperature, i.e. exhibit electrophosphorescence instead of electrofluorescence, which frequently causes an increase in the energy efficiency. Suitable for this purpose are firstly compounds which contain heavy atoms having an atomic number of greater than 36. Particularly suitable compounds are those which contain d- or f-transition metals which satisfy the above-mentioned condition. Very particular preference is given here to corresponding structural units which contain elements from groups 8 to 10 (Ru, Os, Rh, Ir, Pd, Pt). Suitable structural units for the polymers according to the invention here are, for example, various complexes which are described, for example, in WO 02/068435, WO 02/081488, EP 1239526 and WO 04/026886. Corresponding monomers are described in WO 02/068435 and WO 2005/042548 A1.

Preferred units from group 6 are those of the following formulae:

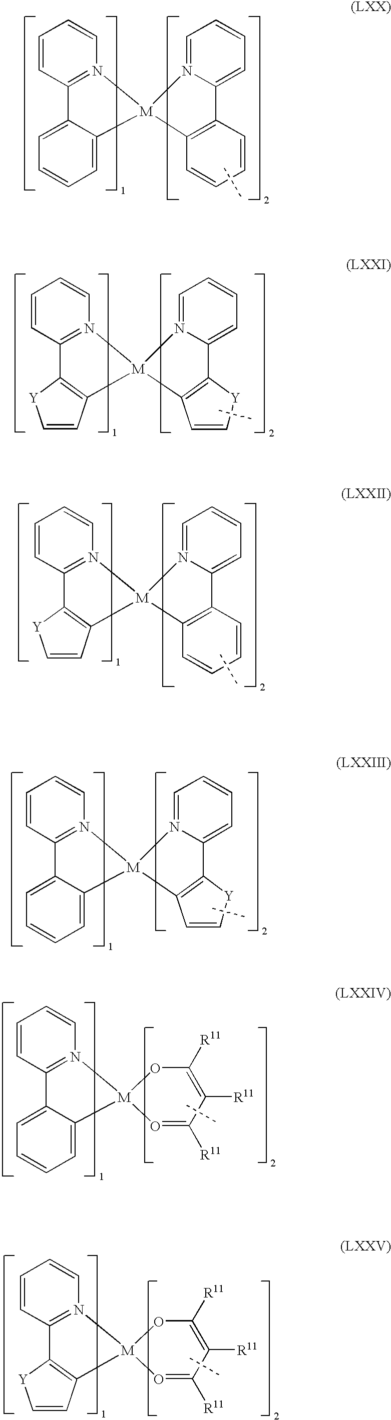

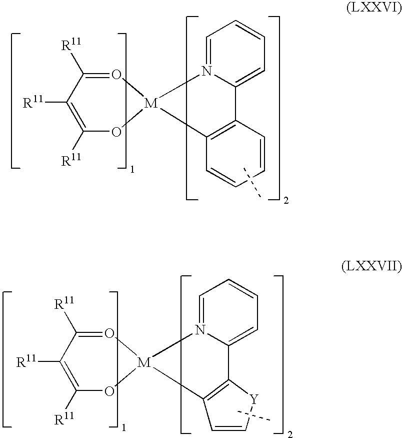

in which M stands for Rh or Ir, Y has the above-mentioned meaning, and the various formulae may be substituted in the free positions by one or more substituents R¹¹ as defined above.
Group 7—Structural Units which Improve the Transfer from the Singlet State to the Triplet State:

These are, in particular, those units which improve the transfer from the singlet state to the triplet state and which, employed in support of the structural elements from group 6, improve the phosphorescence properties of these structural elements. Suitable for this purpose are, in particular, carbazole and bridged carbazole dimer units, as described, for example, in WO 04/070772 and WO 04/113468. Also suitable for this purpose are ketones, phosphine oxides, sulfoxides and similar compounds, as described, for example, in WO 2005/040302 A1.

It is also possible for more than one structural unit from one of groups 1 to 7 to be present simultaneously.

The polymer according to the invention may furthermore likewise contain metal complexes, which are generally built up from one or more ligands and one or more metal centres, bonded into the main or side chain.

Preference is given to polymers according to the invention which at the same time, besides structural units of the formula (1), additionally also contain one or more units selected from groups 1 to 7.

Preference is given here to polymers according to the invention which, besides units of the formula (1), also contain units from group 1, particularly preferably at least 1 mol % of these units.

It is likewise preferred for the polymers according to the invention to contain units which improve the charge transport or charge injection, i.e. units from group 2 and/or 3; a proportion of 1 to 30 mol % of these units is particularly preferred; a proportion of 2 to 10 mol % of these units is very particularly preferred.

It is furthermore particularly preferred for the polymers according to the invention to contain units from group 1, units from group 2 and/or 3, and units from group 5.

The proportion of the units of the formula (1) is preferably at least 10 mol %, particularly preferably at least 30 mol %, in particular at least 50 mol %. This preference applies in particular if the units of the formula (1) are the polymer backbone. In the case of other functions, other proportions may be preferred, for example a proportion in the order of 5 to 20 mol % in the case of the hole conductor or emitter in an electroluminescent polymer. For other applications, for example for organic transistors, the preferred proportion may again be different, for example up to 100 mol % in the case of hole- or electron-conducting units.

The polymers according to the invention preferably have 10 to 10,000, particularly preferably 20 to 5000 and in particular 50 to 2000 recurring units. Corresponding dendrimers may also have fewer recurring units.

The requisite solubility of the polymers and dendrimers is ensured, in particular, by the substituents on the various recurring units, both by substituents R¹⁻⁴ on units of the formula (1) and also by substituents on the other recurring units.

The polymers according to the invention are either homopolymers comprising units of the formula (1) or copolymers. The polymers according to the invention may be linear or branched (crosslinked). Besides one or more structures of the formula (1), or preferred sub-formulae thereof, copolymers according to the invention can potentially have one or more further structures from groups 1 to 4 mentioned above.

The copolymers according to the invention may have random, alternating or block-like structures or also have a plurality of these structures in an alternating arrangement. The way in which copolymers having block-like structures can be obtained and which further structural elements are particularly preferred for this purpose are described in detail, for example, in WO 2005/014688. This specification is incorporated into the present application by way of reference. It should likewise be re-emphasised at this point that the polymer may also have dendritic structures.

The polymers according to the invention are generally prepared by polymerisation of one or more types of monomer, at least one of which is described by the formula (1). Suitable polymerisation reactions are known to the person skilled in the art and are described in the literature. Particularly suitable and preferred polymerisation and coupling reactions, all of which result in C—C linkages, are the SUZUKI, YAMAMOTO, STILLE, HECK, NEGISHI, SONOGASHIRA or HIYAMA reactions.

The way in which the polymerisation can be carried out by these methods and the way in which the polymers can then be separated off from the reaction medium and purified are known to the person skilled in the art and are described in detail in the literature, for example in WO 2003/048225 and WO 2004/037887.

The C—C linking reactions are preferably selected from the groups of the SUZUKI coupling, the YAMAMOTO coupling and the STILLE coupling.

The dendrimers according to the invention can be prepared by processes known to the person skilled in the art or analogously thereto. Suitable processes are described in the literature, such as, for example, in Frechet, Jean M. J.; Hawker, Craig J., “Hyperbranched polyphenylene and hyperbranched polyesters: new soluble, three-dimensional, reactive polymers”, Reactive & Functional Polymers (1995), 26 (1-3), 127-36; Janssen, H. M.; Meijer, E. W., “The synthesis and characterization of dendritic molecules”, Materials Science and Technology (1999), 20 (Synthesis of Polymers), 403-458; Tomalia, Donald A., “Dendrimer molecules”, Scientific American (1995), 272 (5), 62-6, WO 02/67343 A1 and WO 2005/026144 A1.

For the synthesis of the polymers and dendrimers, the corresponding monomers are required. The synthesis of units from groups 1 to 7 is known to the person skilled in the art and is described in the literature, for example in WO 2005/014689. This and the literature cited therein are incorporated into the present application by way of reference.

Monomers which lead to structural units of the formula (1) in polymers and dendrimers according to the invention are preferably selected from formula (1)

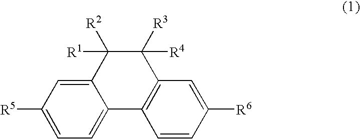

in which R¹⁻⁴ have the meanings indicated above, and R⁵ and R⁶ each, independently of one another, denote a reactive group Z which is suitable for a polymerisation reaction.

Particularly preferred groups Z are selected from halogen, in particular Cl, Br, I, O-tosylate, O-triflate, O—SO₂R′, B(OH)₂, B(OR′)₂ or Sn(R′)₃, furthermore O-mesylate, O-nonaflate, SiMe₂F, SiMeF₂, CR′═C(R′)₂ or C≡CH, in which R′ denotes optionally substituted alkyl or aryl, and two groups R′ may form an aromatic or aliphatic, mono- or polycyclic ring system. “Aryl” and “alkyl” preferably have one of the meanings indicated above.

Preference is furthermore given to monomers of the sub-formulae (1a)-(1g) shown above in which R⁵ and R⁶ each, independently of one another, denote Z.

The present invention likewise relates to novel monomers which lead to units of the formula (1) in the polymer and dendrimer, in particular novel monomers of the formula (1) and the sub-formulae (1a) to (1g).

The monomers can be prepared by processes which are known to the person skilled in the art and are described in standard works of organic chemistry. Particularly suitable and preferred processes are described in the examples.

The polymers according to the invention have the following advantages over the polymers in accordance with the prior art:
(1) The polymers according to the invention exhibit higher photostability compared with polymers in accordance with the prior art. This is of crucial importance for use of these polymers since they must not be decomposed either by the radiation liberated by electroluminescence or by externally incident radiation. This property is still unsatisfactory in the case of polymers in accordance with the prior art.
(2) The polymers according to the invention have (with an otherwise identical or similar composition) comparable or higher luminous efficiencies in the application. This is of enormous importance since thus either the same brightness can be achieved with lower energy consumption, which is very important, in particular, in mobile applications (displays for mobile phones, pagers, PDAs, etc.) which rely on batteries. Conversely, higher brightnesses are obtained with the same energy consumption, which may be interesting, for example, for illumination applications.
(3) Furthermore, it has surprisingly been found that, again in direct comparison, the polymers according to the invention have comparable or longer operating lifetimes.
(4) The polymers according to the invention and solutions and formulations comprising them have improved processability, in particular lower viscosity in solution.
(5) The polymers according to the invention exhibit greater colour stability, in particular in the case of dark-blue colour coordinates.

It may additionally be preferred to use the polymer according to the invention not as the pure substance, but instead as a mixture (blend) together with further polymeric, oligomeric, dendritic or low-molecular-weight substances of any desired type. These may, for example, improve the electronic properties or emit themselves. The present invention therefore also relates to blends of this type.

The invention furthermore relates to solutions and formulations comprising one or more polymers or blends according to the invention in one or more solvents. The way in which polymer solutions can be prepared is known to the person skilled in the art and is described, for example, in WO 02/072714, WO 03/019694 and the literature cited therein.

These solutions can be used in order to produce thin polymer layers, for example by area-coating methods (for example spin coating) or by printing processes (for example ink-jet printing).

The polymers according to the invention can be used in PLEDs. The way in which PLEDs can be produced is known to the person skilled in the art and is described in detail, for example, as a general process in WO 2004/070772, which should be adapted correspondingly for the individual case.

As described above, the polymers according to the invention are very particularly suitable as electroluminescent materials in PLEDs or displays produced in this way.

For the purposes of the invention, electroluminescent materials are taken to mean materials which can be used as active layer in a PLED. Active layer means that the layer is capable of emitting light on application of an electric field (light-emitting layer) and/or that it improves the injection and/or transport of the positive and/or negative charges (charge-injection or charge-transport layer).

The invention therefore also relates to the use of a polymer or blend according to the invention in a PLED, in particular as electroluminescent material.

The invention thus likewise relates to a PLED having one or more active layers, where at least one of these active layers comprises one or more polymers according to the invention. The active layer can be, for example, a light-emitting layer and/or a transport layer and/or a charge-injection layer. The polymers according to the invention are particularly preferably used in PLEDs having an interlayer.

The present application text and also the examples below are directed to the use of polymers or blends according to the invention in relation to PLEDs and corresponding displays. In spite of this restriction of the description, it is possible for the person skilled in the art, without further inventive step, also to use the polymers according to the invention as semiconductors for further uses in other electronic devices, for example in organic field-effect transistors (O-FETs), in organic integrated circuits (O-ICs), in organic thin-film transistors (O-TFTs), in organic solar cells (O-SCs), in organic laser diodes (O-lasers) or in organic photovoltaic (OPV) elements or devices, to mention but a few applications.

The present invention likewise relates to the use of the polymers according to the invention in the corresponding devices.

It is likewise easy for the person skilled in the art to apply the descriptions given above for conjugated polymers to conjugated dendrimers without further inventive step. The present invention thus also relates to conjugated dendrimers of this type.

The compounds of the formula (1) can be prepared by methods known to the person skilled in the art and described in the literature, such as, for example, in WO 2005/014689. Further suitable and preferred synthetic processes are given in the examples. The invention furthermore relates to the synthetic processes described above and below for monomers and polymers according to the invention. Monomers of the formula (1) can be prepared, for example, by reacting a 2,7-dihalophenanthrene-9,10-diquinone with an organomagnesium halide by the Grignard method, heating the resultant 2,7-dihalo-9,10-disubstituted 9,10-dihydrophenanthrene-9,10-diol with a preferably strong acid, and reacting the resultant 2,7-dihalo-10,10-disubstituted 10-hydrophenanthren-9-one with an organometallic zinc reagent.

The following examples are intended to explain the invention without restricting it. In particular, the features, properties and advantages described therein of the defined compounds on which the particular example is based can also be applied to other compounds which are not indicated in detail, but fall within the scope of protection of the claims, unless stated otherwise elsewhere.

### EXAMPLE 1

### Synthesis of a Monomer

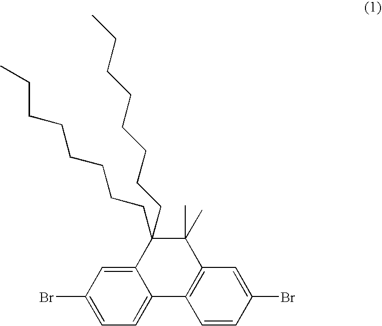

Monomer (1) is prepared as described below.

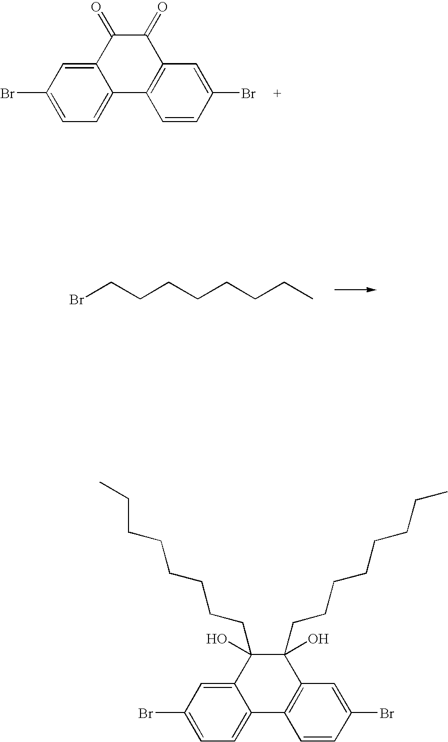

50 g of Mg are initially introduced, the apparatus is dried by heating, and 358 ml of octyl bromide dissolved in 700 ml of dry THF are added dropwise. The solution is added dropwise at such a rate that the reaction refluxes without heating. When the addition is complete (after about 40 minutes), an oil bath preheated to 85° C. is placed under the apparatus, and the mixture is refluxed for about a further 1.5 hours until the Mg has completely dissolved. The oil bath is removed, and 1.3 l of dry THF are added, and the mixture is cooled to RT and transferred by means of a funnel and spout under argon into the dropping funnel of a second apparatus which has been dried by heating.

In the 2nd apparatus, 253 g of dibromophenanthrenequinone are suspended in 1000 ml of THF. The suspension is cooled to about 0° C., and the Grignard solution is added dropwise at such a rate that the internal temperature does not exceed 25° C., and the mixture is subsequently stirred overnight at room temperature. 320 ml of glacial acetic acid/H₂O 1:1 are added dropwise over the course of 20 minutes with ice-cooling; this reaction is highly exothermic. The mixture is stirred for a further hour, during which two phases form. The phases are separated, and the organic phase is reduced to 0.5 l in vacuo. The organic phase is subsequently diluted with 1.5 l of ethyl acetate and extracted twice with saturated NaCl and dried over Na₂SO₄. The drying agent is filtered off via a fluted filter. The solvent is stripped off in vacuo, giving a dark-red solid. The crude product is recrystallised from 800 ml of heptane. The product obtained is a white solid.

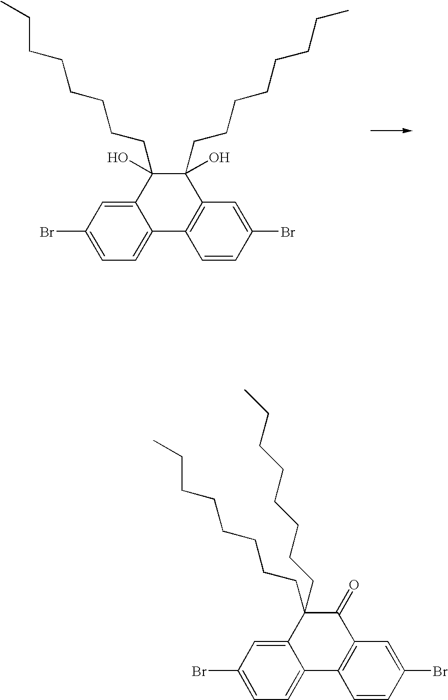

150 g of dioctyldihydroxy-DHP are suspended in 850 ml of glacial acetic acid and 450 ml of trifluoroacetic acid, and the mixture is heated to the boil and stirred under reflux. At an internal temperature of about 60° C., the reaction mixture is a clear yellow solution. After refluxing for about 2.5 hours, a yellow solid precipitates out. The reaction is slowly cooled to room temperature. The precipitate is filtered off with suction and washed with acetic acid and then with water. The solid is washed by stirring overnight at room temperature in about 2 l of water and 1 l of methanol. The fine precipitate is filtered off with suction, washed with water and subsequently with methanol.

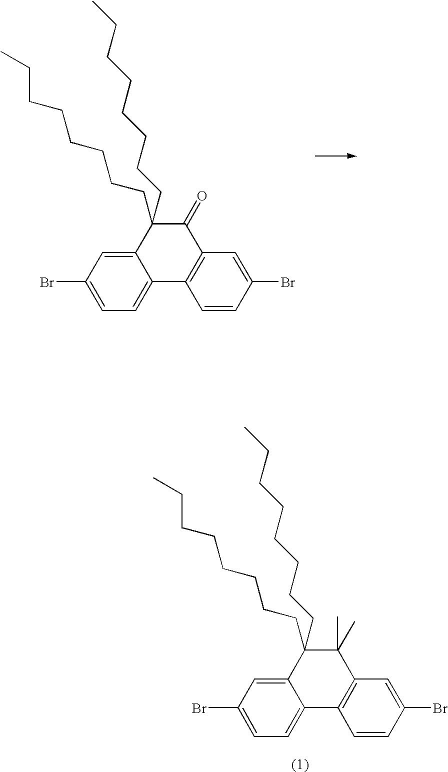

The apparatus is dried by heating under a stream of protective gas and cooled to room temperature. 40 ml of a 1 M titanium tetrachloride solution in dichloromethane are diluted with 63 ml of anhydrous dichloromethane and cooled to −30 to −40° C. in an isopropanol/dry-ice bath. 40 ml of 1 M dimethylzinc solution in heptane are slowly metered in. When the addition is complete, the mixture is stirred for a further 15 minutes. 10 g of the DHP ketone are dissolved in 20 ml of anhydrous dichloromethane and added dropwise to the reaction mixture at −30° C. The reaction mixture is warmed to room temperature overnight. The reaction mixture is carefully added to ice-water. The phases are separated. The organic phase is washed by shaking twice with water, dried over Na₂SO₄, the drying agent is filtered off, and the solvent is stripped off in vacuo. The crude product is chromatographed over a silica-gel column. Eluent:heptane:ethyl acetate 100:1.

### EXAMPLE 2

### Synthesis of Polymers

Polymers P1 to P3 which contain monomers of the compositions below are synthesised by SUZUKI coupling as described in WO 03/048225.

### Composition of Polymers P1 to P3:

Polymer P1:

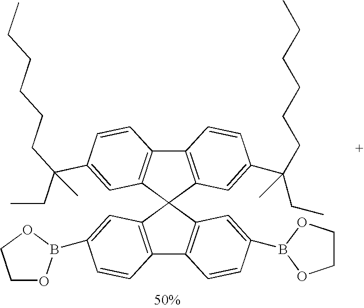

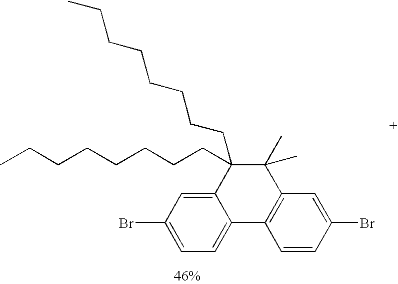

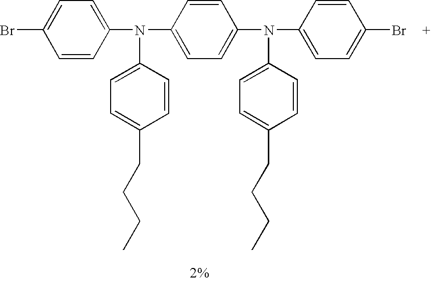

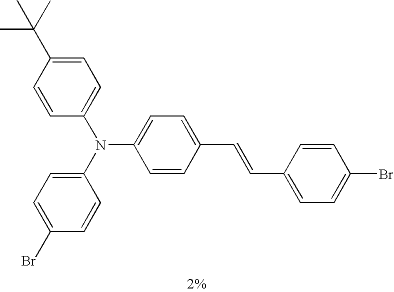

Polymer P2:

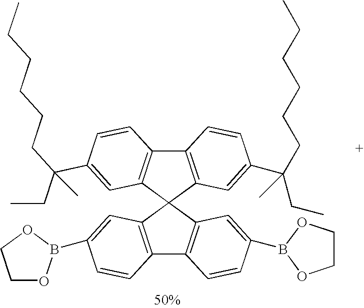

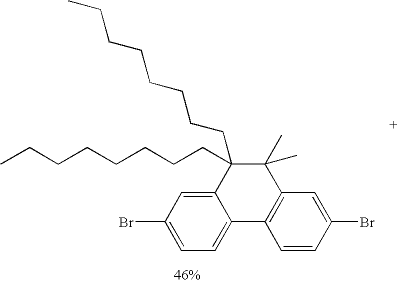

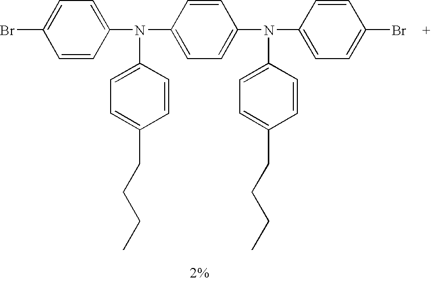

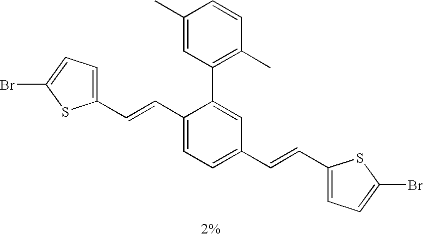

Polymer P3:

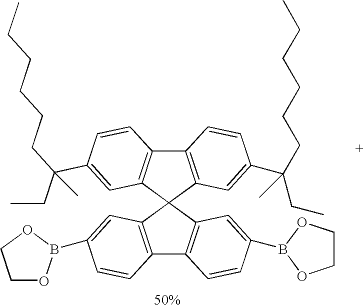

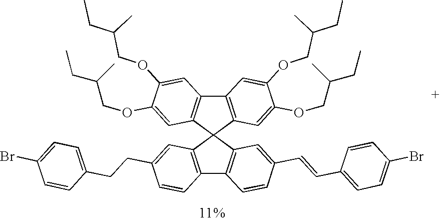

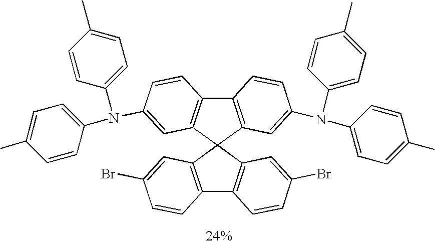

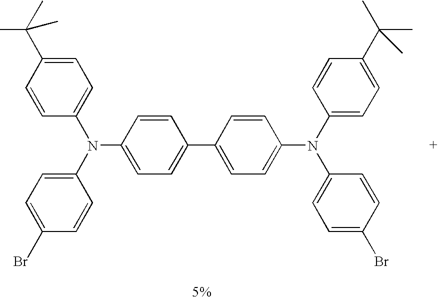

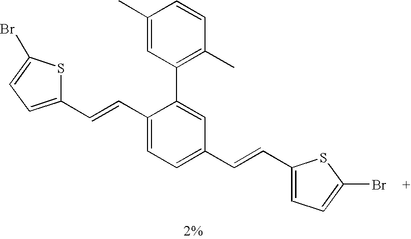

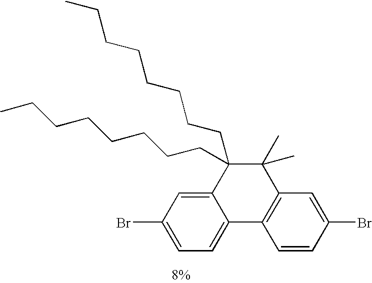

| | | | U@100 | | |
|---|---|---|---|---|---|
| | | Max. eff. | cd/m² | CIE | Lifetime |
| Ex. | Polymer | [Cd/A] | [V] | [x/y] | [h] |
| 1 | P1 | 5.50 | 5.11 | 0.15/0.71 | 194@1000 |
| 2 | P2 | 17.38 | 4.89 | 0.32/0.60 | 159@6000 |
| 3 | P3 | 8.34 | 4.00 | 0.39/0.40 | 427@2000 |

1-18. (canceled)

19. A conjugated polymer or dendrimer, wherein said conjugated polymer or dendrimer comprises one or more units of formula (1) 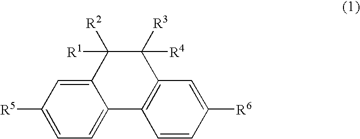
wherein
R¹, R², R³, and R⁴
are, identically or differently on each occurrence, H, F, a straight-chain, branched, or cyclic alkyl, alkenyl, or alkynyl group wherein one or more non-adjacent C atoms are optionally replaced by O, S, CO—O or O—CO—O and wherein one or more H atoms are optionally replaced by fluorine, or an optionally mono- or polysubstituted aryl, aralkyl, aralkenyl, aralkynyl, or heteroaryl group, and wherein two or more radicals R¹, R², R³, and R⁴ optionally define an aliphatic or aromatic, mono- or polycyclic ring system with one another, wherein said aliphatic or aromatic, mono- or polycyclic ring system, together with the dihydrophenanthrene structure, optionally defines a condensed or spiro-linked ring system, and wherein at least two of radicals R¹, R², R³, and R⁴ are different from H; and
R⁵ and R⁶
is a link in said conjugated polymer or dendrimer or a reactive group suitable for a polymerisation reaction.

20. The conjugated polymer or dendrimer of claim 19, wherein units of formula (1) are incorporated into the main chain of said polymer.

21. The conjugated polymer or dendrimer of claim 19, wherein radicals R¹, R², R³, and R⁴ are C₁-C₄₀ alkyl, C₂-C₄₀ alkenyl, C₂-C₄₀ alkynyl, C₃-C₄₀ allyl, C₄-C₄₀ alkyldienyl, C₄-C₄₀ polyenyl, C₆-C₄₀ aryl, C₆-C₄₀ alkylaryl, C₆-C₄₀ arylalkyl, C₆-C₄₀ heteroaryl, C₄-C₄₀ cycloalkyl, or C₄-C₄₀ cycloalkenyl.

22. The conjugated polymer or dendrimer of claim 19, wherein said units of formula (1) are units of formulae (1a), (1b), (1c), (1d), (1e), (1f), and/or (1g): 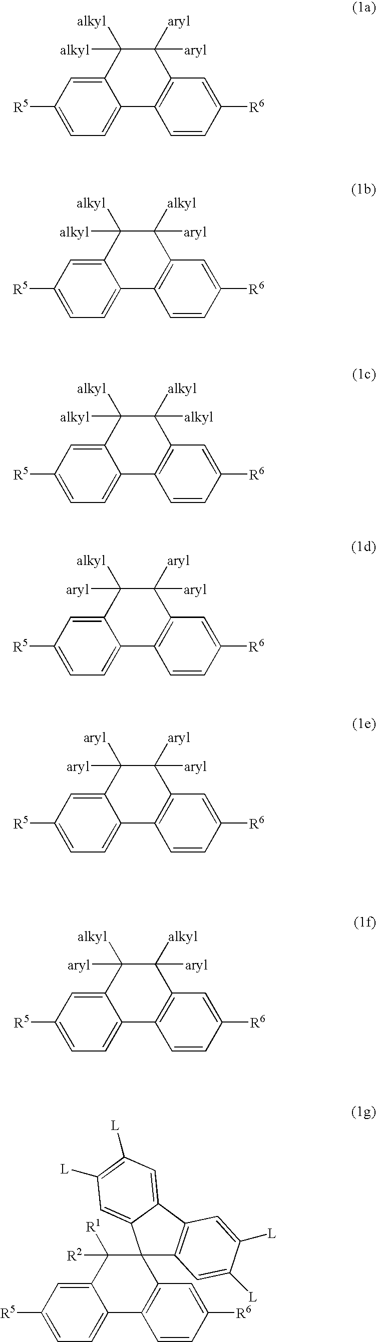
wherein
alkyl is, identically or differently on each occurrence, a straight-chain, branched, or cyclic alkyl radical having 1 to 20 C atoms;
aryl is, identically or differently on each occurrence, an optionally substituted aryl radical having 5 to 20 C atoms or heteroaryl radical having 3 to 20 C atoms;
L is, identically or differently on each occurrence, F, Cl, Br, I, —CN, —NO₂, —NCO, —NCS, —OCN, —SCN, —C(═O)NR₂, —C(═O)X, —C(═O)R, —NR₂, —P(O)R₂, optionally substituted silyl, aryl having 4 to 40 C atoms, or straight-chain or branched alkyl or fluoroalkyl having 1 to 22 C atoms wherein one or more H atoms is optionally be replaced by F or Cl;
X is halogen; and
R is, identically or differently on each occurrence, H, a straight-chain, branched, or cyclic alkyl, alkenyl, alkynyl, or alkoxy chain having 1 to 22 C atoms wherein one or more non-adjacent C atoms is optionally replaced by O, S, CO—O, or O—CO—O and wherein one or more H atoms is optionally replaced by fluorine, or an optionally substituted aryl or aryloxy group having 5 to 40 C atoms wherein one or more C atoms is optionally replaced by O, S, or N.

23. The conjugated polymer or dendrimer of claim 19, wherein said polymer or dendrimer further comprise structural elements selected from the group consisting of fluorenylenes, spirobifluorenylenes, tetrahydropyrenylenes, stilbenzylenes, bisstyrylarylenes, 1,4-phenylenes, 1,4-naphthylenes, 1,4-anthrylenes, 9,10-anthrylenes, 1,6-pyrenylenes, 2,7-pyrenylenes, 4,9-pyrenylenes, 3,9-perylenylenes, 3,10-perylenylenes, 2,7-phenanthrenylenes, 3,6-phenanthrenylenes, 4,4′-biphenylylenes, 4,4″-terphenylylenes, and 4,4′-bi-1,1′-naphthylylenes.

24. The conjugated polymer or dendrimer of claim 19, wherein said polymer or dendrimer further comprise structural elements selected from the group consisting of triarylamines, triarylphosphines, benzidines, tetraarylene-para-phenylenediamines, phenothiazines, phenoxazines, dihydrophenazines, thianthrenes, dibenzo-p-dioxins, phenoxathiynes, carbazoles, azulenes, thiophenes, pyrroles, and furans.

25. The conjugated polymer or dendrimer of claim 19, wherein said polymer or dendrimer further comprise structural elements selected from the group consisting of pyridines, pyrimidines, pyridazines, pyrazines, anthracenes, triarylboranes, oxadiazoles, quinolines, quinoxalines, phenazines, ketones, phosphine oxides, sulfoxides, and triazines.

26. The conjugated polymer or dendrimer of claim 19, wherein the proportion of structural units of formula (1) in said conjugated polymer or dendrimer is in the range of from 1 to 100 mol %.

27. A blend comprising one or more conjugated polymers and/or dendrimers of claim 19 with one or more further polymeric, oligomeric, dendritic, or low-molecular-weight substances.

28. A monomer of formula (1) 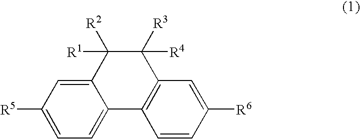
wherein
R¹, R², R³, and R⁴
are, identically or differently on each occurrence, H, F, a straight-chain, branched, or cyclic alkyl, alkenyl, or alkynyl group wherein one or more non-adjacent C atoms are optionally replaced by O, S, CO—O or O—CO—O and wherein one or more H atoms are optionally replaced by fluorine, or an optionally mono- or polysubstituted aryl, aralkyl, aralkenyl, aralkynyl, or heteroaryl group, and wherein two or more radicals R¹, R², R³, and R⁴ optionally define an aliphatic or aromatic, mono- or polycyclic ring system with one another, wherein said aliphatic or aromatic, mono- or polycyclic ring system, together with the dihydrophenanthrene structure, optionally defines a condensed or spiro-linked ring system, and wherein at least two of radicals R¹, R², R³, and R⁴ are different from H; and
R⁵ and R⁶
each, independently of one another, is a reactive group Z suitable for a polymerisation reaction.

29. The monomer of claim 28, wherein Z is, identically or differently on each occurrence, a Cl, Br, I, O-tosylate, O-triflate, O—SO₂R′, B(OH)₂, B(OR′)₂, Sn(R)₃, O-mesylate, O-nonaflate, SiMe₂F, SiMeF₂, CR′═C(R′)₂, or C≡CH, wherein R′ is optionally substituted alkyl or aryl, and wherein two groups R′ optionally define an aromatic or aliphatic, mono- or polycyclic ring system.

30. The monomer of claim 28, wherein said monomer is of formulae (1a), (1b), (1c), (1d), (1e), (1f), or (1g): 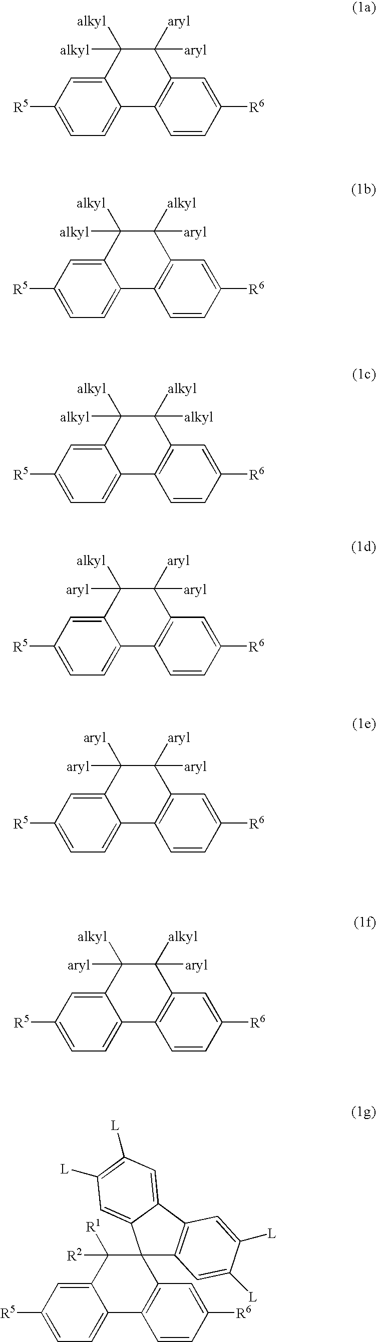
wherein
alkyl is, identically or differently on each occurrence, a straight-chain, branched, or cyclic alkyl radical having 1 to 20 C atoms;
aryl is, identically or differently on each occurrence, an optionally substituted aryl radical having 5 to 20 C atoms or heteroaryl radical having 3 to 20 C atoms;
L is, identically or differently on each occurrence, F, Cl, Br, I, —CN, —NO₂, —NCO, —NCS, —OCN, —SCN, —C(═O)NR₂, —C(═O)X, —C(═O)R, —NR₂, —P(O)R₂, optionally substituted silyl, aryl having 4 to 40 C atoms, or straight-chain or branched alkyl or fluoroalkyl having 1 to 22 C atoms wherein one or more H atoms is optionally be replaced by F or Cl;
X is halogen; and
R is, identically or differently on each occurrence, H, a straight-chain, branched, or cyclic alkyl, alkenyl, alkynyl, or alkoxy chain having 1 to 22 C atoms wherein one or more non-adjacent C atoms is optionally replaced by O, S, CO—O, or O—CO—O and wherein one or more H atoms is optionally replaced by fluorine, or an optionally substituted aryl or aryloxy group having 5 to 40 C atoms wherein one or more C atoms is optionally replaced by O, S, or N.

31. A solution or formulation comprising one or more polymers or dendrimers of claim 19 in one or more solvents.

32. A solution or formulation comprising one or more blends of claim 27 in one or more solvents.

33. A solution or formulation comprising one or more monomers of claim 28 in one or more solvents.

34. An electronic component comprising one or more polymers or dendrimers of claim 19.

35. An electronic component comprising one or more blends of claim 27

36. An electronic component comprising one or more monomers of claim 28.

37. The electronic component of claim 34, wherein said electronic component is a field-effect transistor, organic thin-film transistor, organic integrated circuit, organic solar cell, organic light-emitting diode, organic laser diode, or organic photovoltaic element or device.

38. The electronic component of claim 35, wherein said electronic component is a field-effect transistor, organic thin-film transistor, organic integrated circuit, organic solar cell, organic light-emitting diode, organic laser diode, or organic photovoltaic element or device.

39. The electronic component of claim 36, wherein said electronic component is a field-effect transistor, organic thin-film transistor, organic integrated circuit, organic solar cell, organic light-emitting diode, organic laser diode, or organic photovoltaic element or device.

40. A process for the monomer of claim 28, comprising reacting a 2,7-dihalophenanthrene-9,10-diquinone with an organomagnesium halide by the Grignard method to form a 2,7-dihalo-9,10-disubstituted 9,10-dihydrophenanthrene-9,10-diol, heating said 2,7-dihalo-9,10-disubstituted 9,10-dihydrophenanthrene-9,10-diol with an acid to form a 2,7-dihalo-10,10-disubstituted 10-hydrophenanthren-9-one, and reacting said 2,7-dihalo-10,10-disubstituted 10-hydrophenanthren-9-one with an organometallic zinc reagent.
